(19) United States
(12) Patent Application Publication
(10) Pub. No.: US 2023/0226110 A1
(43) Pub. Date: Jul. 20, 2023

(54) T CELL THERAPY

(71) Applicant: Achilles Therapeutics UK Limited, London (GB)

(72) Inventor: Karl Peggs, London (GB)

(21) Appl. No.: 17/921,240

(22) PCT Filed: Apr. 27, 2021
(86) PCT No.: PCT/GB2021/051015

§ 371 Date: Oct. 25, 2022

(30) Foreign Application Priority Data
Apr. 28, 2020 (GB) 2006221.2
Apr. 9, 2021 (GB) 2105119.8

(51) Int. Cl. A61K 35/17; A61K 38/20; A61K 31/675; A61K 31/7076; A61P 35/00

(52) U.S. Cl. CPC A61K 35/17; A61K 38/2013; A61K 31/675; A61K 31/7076; A61P 35/00

(57) ABSTRACT

The present invention relates to a method of treating cancer in a patient, comprising administering to the patient a T cell therapy and a dose of IL-2 of less than about 2.0 MIU/m2/day.

### FIELD OF THE INVENTION

The present invention relates to a method for treating cancer in a patient using a T cell therapy in combination with a dose of IL-2.

### BACKGROUND

Cancer immunotherapy uses the body's own immune system to target, control and eliminate cancer. One type of cancer immunotherapy is adoptive T cell therapy, whereby antigen-specific T cells are isolated or engineered, expanded ex vivo, and transferred back to patients. The T cells are either derived from the patient themselves (autologous) or from a donor (allogeneic).

T cell therapies rely on enriched or modified human T cells to target and kill cancer cells in a patient. However, transferred T cells typically survive for short periods in vivo and rapidly lose function. One approach to extend the lifespan and function of these introduced T cells is to administer interleukin-2 (IL-2) to recipients of adoptive T cell therapy. However, IL-2 can induce toxicity at high doses and can also expand regulatory T cell populations in vivo, which may reduce the efficacy of adoptive T cell therapies.

### BRIEF DESCRIPTION OF THE INVENTION

The present inventors have now found that lower doses of IL-2 can be used in combination with T cell therapies in order to reduce toxicity and side effects, whilst maintaining intended outcomes. The present invention therefore provides a treatment regimen for T cell therapy in cancer treatment, wherein a low dose of IL-2 is used in combination with the T cell therapy.

Accordingly, the present invention provides a method of treating or preventing cancer in a patient, comprising administering to the patient a T cell therapy and a dose of IL-2 of less than about 2.0 MIU/m²/day.

In one aspect the invention provides a T cell therapy and a dose of IL-2 of less than about 2.0 MIU/m²/day for use in the treatment or prevention of cancer in a patient. In a further aspect the invention provides a T cell therapy for use in the treatment or prevention of cancer in a patient, wherein said T cell therapy is for administration with IL-2, and wherein said IL-2 is for administration at a dose of less than about 2.0 MIU/m²/day.

In one aspect the invention provides a T cell therapy and IL-2 for use in the treatment or prevention of cancer in a patient, wherein said IL-2 is for administration at a dose of less than about 2.0 MIU/m²/day.

In one aspect the invention provides a T cell therapy for use in the manufacture of a medicament for use in the treatment or prevention of cancer, wherein said T cell therapy is for administration with IL-2, wherein said IL-2 is for administration at a dose of less than about 2.0 MIU/m²/day.

In one aspect the invention provides IL-2 for use in the manufacture of a medicament for use in the treatment or prevention of cancer, wherein said IL-2 is for administration in combination with a T cell therapy, wherein said IL-2 is for administration at a dose of less than about 2.0 MIU/m²/day.

In one aspect the invention provides the use of a T cell therapy for the treatment or prevention of cancer, wherein said T cell therapy is for administration with IL-2, wherein said IL-2 is for administration at a dose of less than about 2.0 MIU/m²/day.

In one aspect the invention provides the use of IL-2 for the treatment or prevention of cancer, wherein said IL-2 is for administration with a T cell therapy, wherein said IL-2 is for administration at a dose of less than about 2.0 MIU/m²/day.

The T cell therapy and IL-2 described herein may be for separate, simultaneous or sequential administration to the patient.

### BRIEF DESCRIPTION OF THE FIGURES

FIG. 1 Cell function for patient T-05: Function is measured by cytokine production using flow cytometric analysis. CD3+T cell cytokine production in response to short peptide pools and CD3+T cell cytokine production in response to long peptide pools.

FIG. 2 Tracking cNeT in peripheral circulation allows estimation of the reactive T cell component pre- and post-dosing. RS is the patient rescreening visit, D are visit days post-dosing, W are visit weeks post-dosing. There is detectability of both short (SMP) and long (LMP) peptide master pool reactivity. ELISpot was run in technical triplicates, presented are mean spot forming units (2A). Absolute cell count for B-cells, NK-cells and T-cells were obtained from whole blood TBNK assay and presented as cell count 10⁶/mL blood (2B) and allows for ELISpot mean spot forming unit normalised for the frequency of T-cells per well using TBNK data (2C). 2D shows estimated mean reactive cNeT count/mL in whole blood.

### DETAILED DESCRIPTION OF THE INVENTION

Immunotherapy

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, autologous T cell therapy, tumour-infiltrating lymphocyte (TIL) therapy, engineered T cell therapy, chimeric antigen receptor (CAR) T cell therapy, engineered TCR T cell therapy and allogeneic T cell transplantation. Examples of T cell therapies are described in International Publication Nos, WO2018/002358, WO2013/088114, WO2015/077607, WO2015/143328, WO2017/049166 and WO2011/140170.

The T cells of the immunotherapy may originate from any source known in the art. For example, T cells may be differentiated in vitro from a hematopoietic stem cell population, or T cells can be obtained from a subject. T cells may be obtained from, e.g., peripheral blood mononuclear cells, bone marrow, lymph node tissue, cord blood, thymus tissue, tissue from a site of infection, ascites, pleural effusion, spleen tissue, and tumours. In addition, the T cells may be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

In one aspect of the invention as described herein, a single dose of T cell therapy is administered to the patient. In one aspect a single dose of T cell therapy is administered to the patient on day 0 only. In other aspects of the invention, multiple doses of T cell therapy are administered to the patient starting from day 0. For example, the number of doses of T cell therapy may be 2, 3, 4, 5, 6, 7, 8, 9, 10 or more than 10 doses.

Dosing may be once, twice, three times, four times, five times, six times, or more than six times per year. Alternatively, dosing may be once, twice, three times, four times, five times, six times, or more than six times per month. In a further aspect dosing may be once, twice, three times, four times, five times, six times, or more than six times every two weeks. In yet a further aspect dosing may be once, twice, three times, four times, five times, six times, or more than six times per week, for example once a week, or once every other day.

Administration of the T cell therapy may continue as long as necessary.

In one aspect the T cell therapy may comprise CD8+ T cells, CD4+ T cells or CD8+ and CD4+ T cells.

The T cell therapy as described herein may be used in vitro, ex vivo or in vivo, for example either for in situ treatment or for ex vivo treatment followed by the administration of the treated cells to the body.

In certain aspects according to the invention as described herein the T cell therapy is reinfused into a subject, for example following T cell isolation and expansion as described herein. Suitable methods for generating, selecting, expanding and reinfusing T cells are known in the art.

The T cell therapy may be administered to a subject at a suitable dose. The dosage regimen may be determined by the attending physician and clinical factors. It is accepted in the art that dosages for any one patient depend upon many factors, including the patient's size, body surface area, age, the particular compound to be administered, sex, time and route of administration, general health, and other drugs being administered concurrently.

The T cell therapy may involve the transfer of a given number of T cells as described herein to a patient, for example TILs or CAR-T cells. The therapeutically effective amount of T cells may be at least about 10³ cells, at least about 10⁴ cells, at least about 10⁵ cells, at least about 10⁶ cells, at least about 10⁷ cells, at least about 10⁸ cells, at least about 10⁹ cells, at least about 10¹⁰ cells, at least about 10¹¹ cells, at least about 10¹² or at least about 10¹³ cells.

Other suitable doses of T cells may be as described in, for example, WO 2016/191755, WO2019/112932, WO2018/226714, WO2018/182817, WO2018/129332, WO2018/129336, WO2018/094167, WO2018/081789 and WO2018/081473.

Tumour-Infiltrating Lymphocyte (TIL) Therapy

In one aspect of the invention the T cell therapy uses TILs.

Tumour-infiltrating lymphocyte (TIL) immunotherapy is a type of adoptive T cell therapy wherein T cells that have infiltrated tumour tissue are isolated, enriched in vitro and administered to a patient. Generation of TIL cultures may be performed by first culturing resected tumour fragments or tumour single-cell suspensions in medium containing IL-2. This initial pre-expansion may be followed by a rapid expansion protocol (REP) involving the activation of TILs using an anti-CD3 monoclonal antibody in the presence of irradiated peripheral blood mononuclear cells (PBMC) and IL-2. Examples of TIL therapies and expansion protocols are described in International Patent Publication Nos. WO2018/081473, WO2018/081789, WO2018/094167, WO2018/129336, WO2018/129332, WO2018/182817, WO2018/226714, WO2019/100023, WO2019/112932 and US granted patent Nos. U.S. Pat. Nos. 8,383,099 and 9,074,185.

Engineered T Cell Therapy

In one aspect of the invention the T cell therapy uses engineered T cells. The T cells are isolated from the patient (e.g. from a blood sample) and are modified, for example to express a chimeric antigen receptor (CAR) or a TCR receptor that binds to a target antigen.

CARs are proteins which, in their usual format, graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals.

The most common form of these molecules use single-chain variable fragments (scFv) derived from monoclonal antibodies to recognize a target antigen. The scFv is fused via a spacer and a transmembrane domain to a signalling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Affinity-enhanced TCRs are generated by identifying a T cell clone from which the TCR a and β chains with the desired target specificity are cloned. The candidate TCR then undergoes PCR directed mutagenesis at the complimentary determining regions of the α and β chains. The mutations in each CDR region are screened to select for mutants with enhanced affinity over the native TCR. Once complete, lead candidates are cloned into vectors to allow functional testing in T cells expressing the affinity-enhanced TCR.

T cells may bear high affinity TCRs, and hence affinity enhancement may not be necessary. High affinity TCRs may be isolated from T cells from a subject and may not require affinity enhancement.

Identified TCRs and/or CARs may be expressed in autologous T cells from a subject using methods which are known in the art, for example by introducing DNA or RNA coding for the TCR or CAR by one of many means including transduction with a viral vector, transfection with DNA or RNA.

Antigens

In one aspect of the invention the T cell therapy comprises T cells which target cancer-associated or tumour-specific antigens.

Tumour antigens include the following: CEA, immature laminin receptor, TAG-72, HPV E6 and E7, BING-4, calcium-activated chloride channel 2, cyclin-B1, 9D7, Ep-CAM, EphA3, Her2/neu, telomerase, mesothelin, SAP-1, survivin, BAGE family, CAGE family, GAGE family, MAGE family, SAGE family, XAGE family, NY-ESO-1/LAGE-1, PRAME, SSX-2, Melan-A/MART-1, gp100/pmel17, tyrosinase, TRP-1/-2, P. polypeptide, MC1R, prostate-specific antigen, beta-catenin, BRCA1/2, CDK4, CML66, fibronectin, MART-2, p53, ras, TGF-betaRII and MUC1.

Tumour antigens may also include the following: 707-AP=707 alanine proline, AFP=alpha (α)-fetoprotein, ART-4=adenocarcinoma antigen recognized by T cells 4, BAGE=B antigen; β-catenin/m, β-catenin/mutated, Bcr-abl=breakpoint clusterregion-Abelson, CAMEL=CTL-recognized antigen on melanoma, CAP-1=carcinoembryonic antigen peptide-1, CASP-8=caspase-8, CDC27m=cell-division-cycle 27 mutated, CDK4/m=cycline-dependent kinase 4 mutated, CEA=carcinoembryonic antigen, CT=cancer/testis (antigen), Cyp-B=cyclophilin B, DAM=differentiation antigen melanoma (the epitopes of DAM-6 and DAM-10 are equivalent, but the gene sequences are different. DAM-6 is also called MAGE-B2 and DAM-10 is also called MAGE-B1), ELF2M=elongation factor 2 mutated, ETV6-AML1=Etsvariant gene 6/acute myeloid leukemia 1 gene ETS, G250=glycoprotein 250, GAGE=G antigen, GnT-V=N-acetylglucosaminyltransferase V, Gp100=glycoprotein 100 kD, HAGE=helicose antigen, HER-2/neu=human epidermal receptor-2/neurological, HLA-A*0201-R170I=arginine (R) to isoleucine (I) exchange at residue 170 of the α-helix of the α2-domain in the HLA-A2 gene, HPV-E7=human papilloma virus E7, HSP70-2M=heat shock protein 70-2 mutated, HST-2=human signet ring tumor-2, hTERT or hTRT=human telomerase reverse transcriptase, iCE=intestinal carboxylesterase, KIAA0205=name of the gene as it appears in databases, LAGE=L antigen, LDLR/FUT=low density lipid receptor/GDP-L-fucose: β-D-galactosidase 2-α-L-fucosyltransferase, MAGE=melanoma antigen, MART-1/Melan-A=melanomaantigen recognized by T cells-1/Melanoma antigen A, MC1R=melanocortin 1 receptor, Myosin/m=myosin mutated üMUC1=mucin 1, MUM-1, -2, -3=melanomaubiquitous mutated 1, 2, 3, NA88-A=NA cDNA clone of patient M88, NY-ESO-1=New York—esophageous 1, P15=protein 15, p190 minor bcr-abl=protein of 190 3 KD bcr-abl, Pml/RARα=promyelocytic leukaemia/retinoic acid receptor α, PRAME=preferentially expressed antigen of melanoma, PSA=prostate-specific antigen, PSM=prostate-specific membrane antigen, RAGE=renal antigen, RU1 or RU2=renalubiquitous 1 or 2, SAGE=sarcoma antigen, SART-1 or SART-3=squamous antigenrejecting tumor 1 or 3, TEL/AML1=translocation Ets-family leukemia/acute myeloidleukemia 1, TPI/m=triosephosphate isomerase mutated, TRP-1=tyrosinase relatedprotein 1, or gp75, TRP-2=tyrosinase related protein 2, TRP-2/INT2=TRP-2/intron2, WT1=Wlms' tumor gene.

Neoantigens

In one aspect of the invention the antigen may be a neoantigen.

A “neoantigen” is a tumour-specific antigen which arises as a consequence of a mutation within a cancer cell. Thus, a neoantigen is not expressed (or expressed at a significantly lower level) by healthy (i.e. non-tumour) cells in a subject. A neoantigen may be processed to generate distinct peptides which can be recognised by T cells when presented in the context of MHC molecules. As described herein, neoantigens may be used as the basis for cancer immunotherapies. References herein to “neoantigens” are intended to include also peptides derived from neoantigens. The term “neoantigen” as used herein is intended to encompass any part of a neoantigen that is immunogenic. An “antigenic” molecule as referred to herein is a molecule which itself, or a part thereof, is capable of stimulating an immune response, when presented to the immune system or immune cells in an appropriate manner. The binding of a neoantigen to a particular MHC molecule (encoded by a particular HLA allele) may be predicted using methods which are known in the art. Examples of methods for predicting MHC binding include those described by Lundegaard et al., O'Donnel et al., and Bullik-Sullivan et al. For example, MHC binding of neoantigens may be predicted using the netMHC-3 (Lundegaard et al.) and netMHCpan4 (Jurtz et al.) algorithms. A neoantigen that has been predicted to bind to a particular MHC molecule is thereby predicted to be presented by said MHC molecule on the cell surface.

The neoantigen described herein may be caused by any non-silent mutation which alters a protein when expressed by a cancer cell compared to the non-mutated protein expressed by a wild-type, healthy cell. In other words, the mutation results in the expression of an amino acid sequence that is not expressed, or expressed at a very low level in a wild-type, healthy cell. For example, the mutation may occur in the coding sequence of a protein, thus altering the amino acid sequence of the resulting protein. This may be referred to as a “coding mutation”. As another example, the mutation may occur in a splice site, thus resulting in the production of a protein that contains a set of exons that is different or less common in the wild type protein. As a further example, the mutated protein may be a translocation or fusion.

A “mutation” refers to a difference in a nucleotide sequence (e.g. DNA or RNA) in a tumour cell compared to a healthy cell from the same individual. The difference in the nucleotide sequence can result in the expression of a protein which is not expressed by a healthy cell from the same individual. For example, the mutation may be one or more of a single nucleotide variant (SNV), a multiple nucleotide variant (MNV), a deletion mutation, an insertion mutation, an indel mutation, a frameshift mutation, a translocation, a missense mutation, a splice site mutation, a fusion, or any other change in the genetic material of a tumour cell.

An “indel mutation” refers to an insertion and/or deletion of bases in a nucleotide sequence (e.g. DNA or RNA) of an organism. Typically, the indel mutation occurs in the DNA, preferably the genomic DNA, of an organism. In embodiments, the indel may be from 1 to 100 bases, for example 1 to 90, 1 to 50, 1 to 23 or 1 to 10 bases. An indel mutation may be a frameshift indel mutation. A frameshift indel mutation is an insertion or deletion of one or more nucleotides that causes a change in the reading frame of the nucleotide sequence. Such frameshift indel mutations may generate a novel open-reading frame which is typically highly distinct from the polypeptide encoded by the non-mutated DNA/RNA in a corresponding healthy cell in the subject.

The mutations may be identified by exome sequencing, RNA-seq, whole genome sequencing and/or targeted gene panel sequencing and/or routine Sanger sequencing of single genes. Suitable methods are known in the art. Descriptions of exome sequencing and RNA-seq are provided by Boa et al. (Cancer Informatics. 2014; 13(Suppl 2):67-82.) and Ares et al. (Cold Spring Harb Protoc. 2014 Nov. 3; 2014(11):1139-48); respectively. Descriptions of targeted gene panel sequencing can be found in, for example, Kammermeier et al. (J Med Genet. 2014 November; 51(11):748-55) and Yap K L et al. (Clin Cancer Res. 2014. 20:6605). See also Meyerson et al., Nat. Rev. Genetics, 2010 and Mardis, Annu Rev Anal Chem, 2013. Targeted gene sequencing panels are also commercially available (e.g. as summarised by Biocompare ((http://www.biocompare.com/Editorial-Articles/161194-Build-Your-Own-Gene-Panels-with-These-Custom-NGS-Targeting-Tools/)).

Sequence alignment to identify nucleotide differences (e.g. SNVs) in DNA and/or RNA from a tumour sample compared to DNA and/or RNA from a non-tumour sample may be performed using methods which are known in the art. For example, nucleotide differences compared to a reference sample may be performed using the method described by Koboldt et al. (Genome Res. 2012; 22: 568-576). The reference sample may be the germline DNA and/or RNA sequence.

Clonal Neoantigens

In one aspect the neoantigen may be a clonal neoantigen.

A “clonal neoantigen” (also sometimes referred to as a “truncal neoantigen”) is a neoantigen arising from a clonal mutation. A “clonal mutation” (sometimes referred to as a “truncal mutation”) is a mutation that is present in essentially every tumour cell in one or more samples from a subject (or that can be assumed to be present in essentially every tumour cell from which the tumour genetic material in the sample(s) is derived). Thus, a clonal mutation may be a mutation that is present in every tumour cell in one or more samples from a subject. For example, a clonal mutation may be a mutation which occurs early in tumorigenesis.

A “subclonal neoantigen” (also sometimes referred to as a “branched neoantigen”) is a neoantigen arising from a subclonal mutation. A “subclonal mutation” (also sometimes referred to as a “branch mutation”) is a mutation that is present in a subset or a proportion of cells in one or more tumour samples from a subject (or that can be assumed to be present in a subset of the tumour cells from which the tumour genetic material in the sample(s) is derived). For example, a subclonal mutation may be the result of a mutation occurring in a particular tumour cell later in tumorigenesis, which is found only in cells descended from that cell.

The wording “essentially every tumour cell” in relation to one or more samples of a subject may refer to at least 70%, at least 75%, at least 80%, at least 85%, at least 90%, at least 91%, at least 92%, at least 93%, at least 94% at least 95%, at least 96%, at least 97%, at least 98%, or at least 99% of the tumour cells in the one or more samples or the subject.

As such, a clonal neoantigen is a neoantigen which is expressed effectively throughout a tumour. A subclonal neoantigen is a neoantigen that is expressed in a subset or a proportion of cells or regions in a tumour. ‘Expressed effectively throughout a tumour’ may mean that the clonal neoantigen is expressed in all regions of the tumour from which samples are analysed.

It will be appreciated that a determination that a mutation is ‘encoded (or expressed) within essentially every tumour cell’ refers to a statistical calculation and is therefore subject to statistical analysis and thresholds.

Likewise, a determination that a clonal neoantigen is ‘expressed effectively throughout a tumour’ refers to a statistical calculation and is therefore subject to statistical analysis and thresholds.

Various methods for determining whether a neoantigen is “clonal” are known in the art. Any suitable method may be used to identify a clonal neoantigen.

By way of example, the cancer cell fraction (CCF), describing the proportion of cancer cells that harbour a mutation, may be used to determine whether mutations are clonal or subclonal. For example, the cancer cell fraction may be determined by integrating variant allele frequencies with copy numbers and purity estimates as described by Landau et al. (Cell. 2013 Feb. 14; 152(4):714-26).

Suitably, CCF values may be calculated for all mutations identified within each and every tumour region analysed. If only one region is used (i.e. only a single sample), only one set of CCF values will be obtained. This will provide information as to which mutations are present in all tumour cells within that tumour region and will thereby provide an indication if the mutation is clonal or subclonal.

Such a CCF estimate can also be used to identify mutations that are likely to be clonal. A clonal mutation may be defined as a mutation which has a cancer cell fraction (CCF) 0.75, such as a CCF 0.80, 0.85. 0.90, 0.95 or 1.0. A subclonal mutation may be defined as a mutation which has a CCF<0.95, 0.90, 0.85, 0.80, or 0.75. In one aspect, a clonal mutation is defined as a mutation which has a CCF≥0.95 and a subclonal mutation is defined as a mutation which has a CCF<0.95.

As stated, determining a clonal mutation is subject to statistical analysis and threshold. A CCF estimate may be associated with (e.g. derived from) a distribution associating a probability with each of a plurality of possible values of CCF between 0 and 1, from which statistical estimates of confidence may be obtained. For example, a mutation may be defined as likely to be a clonal mutation if the 95% CCF confidence interval is >=0.75, i.e. the upper bound of the 95% confidence interval of the estimated CCF is greater than or equal to 0.75. In other words, a mutation may be defined as likely to be a clonal mutation if there is an interval of CCF with lower bound L and upper bound H that is such that P(L<CCF<H)=95% with H>=0.75. In other words, a mutation may be identified as clonal if P(CCF>0.75)>=0.5.

In one aspect a mutation may be defined as a clonal mutation if the 95% confidence interval of the CCF includes CCF=1.

In another aspect a mutation may be identified as clonal if there is more than a 50% chance or probability that its cancer cell fraction (CCF) reaches or exceeds the required value as defined above, for example 0.75 or 0.95, such as a chance or probability of 55%, 60%, 65%, 70%, 75%, 80%, 85%, 90%, 95% or more.

Probability values may be expressed as percentages or fractions. The probability may be defined as a posterior probability.

In one aspect, a mutation may be identified as clonal if the probability that the mutation has a cancer cell fraction greater than 0.95 is 0.75.

In another aspect, a mutation may be identified as clonal if there is more than a 50% chance that its cancer cell fraction (CCF) is 0.95.

In a further aspect, mutations may be classified as clonal or subclonal based on whether the posterior probability that their CCF exceeds a first threshold (e.g. 0.95) is greater or lesser than a second threshold (e.g. 0.5), respectively.

In another aspect a mutation may be identified as clonal if the probability that the mutation has a cancer cell fraction greater than 0.75 is 0.5.

In one aspect the T cell therapy may comprise T cells which target a plurality i.e. more than one clonal neoantigen.

In one aspect the number of clonal neoantigens is 2-1000. For example, the number of clonal neoantigens may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, for example the number of clonal neoantigens may be from 2 to 100.

In one aspect, the T cell therapy as described herein may comprise a plurality or population, i.e. more than one, of T cells wherein the plurality of T cells comprises a T cell which recognises a clonal neoantigen and a T cell which recognises a different clonal neoantigen. As such, the T cell therapy comprises a plurality of T cells which recognise different clonal neoantigens.

In one aspect the number of clonal neoantigens recognised by the plurality of T cells is 2-1000. For example, the number of clonal neoantigens recognised may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, for example the number of clonal neoantigens recognised may be from 2 to 100.

In one aspect the plurality of T cells recognises the same clonal neoantigen.

In one aspect the neoantigen may be a subclonal neoantigen as described herein.

As described above, a clonal neoantigen is one which is encoded within essentially every tumour cell, that is the mutation encoding the neoantigen is present within essentially every tumour cell and is expressed effectively throughout the tumour. However, a clonal neoantigen may be predicted to be presented by an HLA molecule encoded by an HLA allele which is lost in at least part of a tumour. In this case, the clonal neoantigen may not actually be presented on essentially every tumour cell. As such, the presentation of the neoantigen may not be clonal, i.e. it is not presented within essentially every tumour cell. Methods for predicting loss of HLA are described in International Patent Publication No. WO2019/012296.

In one aspect of the invention as described herein the neoantigen is predicted to be presented within essentially every tumour cell (i.e. the presentation of the neoantigen is clonal).

Neoantigen-Specific T Cell Therapy

The T cell therapy according to the invention may comprise T cells which target neoantigens. In one aspect of the invention, the T cell therapy may comprise T cells which target clonal neoantigens. In the context of the present invention, the term “target” may mean that the T cell is specific for, and triggers or mounts a response to, the neoantigen.

In one aspect the T cell therapy may comprise T cells which have been selectively expanded to target neoantigens, such as clonal neoantigens.

That is, the T cell therapy may have an increased number of T cells that target one or more neoantigens. For example, the T cell population of the invention will have an increased number of T cells that target a neoantigen compared with the T cells in the sample isolated from the subject. That is to say, the composition of the T cell population will differ from that of a “native” T cell population (i.e. a population that has not undergone the identification and expansion steps discussed herein), in that the percentage or proportion of T cells that target a neoantigen will be increased, and the ratio of T cells in the population that target neoantigens to T cells that do not target neoantigens will be higher in favour of the T cells that target neoantigens.

The T cell population according to the invention may have at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a neoantigen. For example, the T cell population may have about 0.2%-5%, 5%-10%, 10-20%, 20-30%, 30-40%, 40-50%, 50-70% or 70-100% T cells that target a neoantigen. In one aspect the T cell population has at least about 1, 2, 3, 4 or 5% T cells that target a neoantigen, for example at least about 2% or at least 2% T cells that target a neoantigen.

Alternatively put, the T cell population may have not more than about 5, 10, 15, 20, 25, 30, 35, 40, 45, 50, 55, 60, 65, 70, 75, 80, 85, 90, 91, 92, 93, 94, 95, 96, 97, 98, 99, 99.1, 99.2, 99.3, 99.4, 99.5, 99.6, 99.7, 99.8% T cells that do not target a neoantigen. For example, the T cell population may have not more than about 95%-99.8%, 90%-95%, 80-90%, 70-80%, 60-70%, 50-60%, 30-50% or 0-30% T cells that do not target a neoantigen. In one aspect the T cell population has not more than about 99, 98, 97, 96 or 95% T cells that do not target a neoantigen, for example not more than about 98% or 95% T cells that do not target a neoantigen.

An expanded population of neoantigen-reactive T cells may have a higher activity than a population of T cells not expanded, for example, using a neoantigen peptide. Reference to “activity” may represent the response of the T cell population to restimulation with a neoantigen peptide, e.g. a peptide corresponding to the peptide used for expansion, or a mix of neoantigen peptides. Suitable methods for assaying the response are known in the art. For example, cytokine production may be measured (e.g. IL2 or IFNγ production may be measured). The reference to a “higher activity” includes, for example, a 1-5, 5-10, 10-20, 20-50, 50-100, 100-500, 500-1000-fold increase in activity. In one aspect the activity may be more than 1000-fold higher.

The T cell population may be all or primarily composed of CD8+ T cells, or all or primarily composed of a mixture of CD8+ T cells and CD4+ T cells or all or primarily composed of CD4+ T cells.

In particular aspects, the T cells in the T cell therapy may be generated from T cells isolated from a subject with a tumour. The sample may be a tumour sample, a peripheral blood sample (e.g. PBMCs) or a sample from other tissues of the subject.

The T cells may be generated from a sample from the tumour in which the neoantigen is identified. In other words, the T cell population is isolated from a sample derived from the tumour of a patient to be treated. Such T cells are referred to herein as ‘tumour infiltrating lymphocytes’ (TI Ls).

T cells may be isolated using methods which are well known in the art. For example, T cells may be purified from single cell suspensions generated from samples on the basis of expression of CD3, CD4 or CD8. T cells may be enriched from samples by passage through a Ficoll-plaque gradient.

Expansion of T cells may be performed using methods which are known in the art. For example, T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28. The T cells may also be co-cultured with feeder cells, such as peripheral blood mononuclear cells (PBMC) or antigen-presenting cells (APCs). In one aspect, the APCs are irradiated. In another aspect, the APCs are dendritic cells. The dendritic cells may be derived from monocytes obtained from the patient's blood, referred to herein as monocyte-derived dendritic cells (MoDCs).

In one aspect of the invention, T cells that are capable of specifically recognising one or more neoantigens are identified in a sample from the subject and then expanded by ex vivo culture as described herein. Identification of neoantigen-specific T cells in a mixed starting population of T cells may be performed using methods which are known in the art. For example, neoantigen-specific T cells may be identified using MHC multimers comprising a neoantigen peptide.

MHC multimers are oligomeric forms of MHC molecules, designed to identify and isolate T-cells with high affinity to specific antigens amid a large group of unrelated T-cells. Multimers may be used to display class 1 MHC, class 2 MHC, or nonclassical molecules (e.g. CD1d). The most commonly used MHC multimers are tetramers. These are typically produced by biotinylating soluble MHC monomers, which are typically produced recombinantly in eukaryotic or bacterial cells. These monomers then bind to a backbone, such as streptavidin or avidin, creating a tetravalent structure. These backbones are conjugated with fluorochromes to subsequently isolate bound T-cells via flow cytometry, for example.

In another aspect of the invention, the T cells undergo a specific expansion step, whereby T cells that respond to the one or more neoantigens are expanded in preference to other T cells in the starting material that do not respond to the neoantigen(s). This may be achieved by co-culturing the T cells with antigen-presenting cells (APCs) which present the relevant neoantigen(s). The APCs may be pulsed with peptides containing the identified mutations as single stimulants or as pools of stimulating neoantigens or peptides. Alternatively, the APCs may be modified to express the neoantigen sequence(s), for example by transfecting the APCs with mRNA encoding the neoantigen sequence(s).

Other suitable methods for said expansion will be known to those of skill in the art. For example, International Patent Publication No. WO2019/094642 describes a number of protocols for expansion of T cells in response to neoantigens.

T Cell Expansion

In one aspect of the invention, the TIL are expanded by methods that use reduced concentrations of IL-2 in comparison to conventional TIL expansion methods.

By way of example, typical TIL expansion protocols use very high, non-physiological levels of IL-2 in the rapid expansion step. For example, WO 2018/182817 discloses a method of expanding TIL that uses an IL-2 concentration of about 1,000 to about 10,000 IU/ml, for example 3,000 IU/ml of IL-2, in the rapid expansion step.

In contrast, according to the present invention, the T cell therapy may be, or may have been, produced by an expansion method that uses IL-2 at a concentration in the range of from about 10 IU/ml to about 1,000 IU/ml, for example from about 25 IU/ml to about 500 IU/ml, such as from about 50 IU/ml to about 250 IU/ml, preferably from about 75 IU/ml to about 125 IU/ml. The concentration of IL-2 used in a T cell expansion step may therefore be about 10, 25, 50, 75, 100, 125, 150, 200, 250, 300, 400, 500, 600, 700, 800, 900 or 1,000 IU/ml. In one aspect the method may use IL-2 at a concentration of less than about 1,000 IU/ml.

In one aspect the T cells may be pre-expanded, for example prior to co-culture with APCs.

In one embodiment, pre-expanded T cells, for example TIL, may be combined with APCs and co-cultured with IL-2 at a concentration of from 50 IU/ml to 150 IU/ml, preferably about 100 IU/ml, in order to produce the therapeutic T cell product. The IL-2 concentration may remain constant throughout the culture step, for example by controlling the concentration with repeated feeding steps, or may vary throughout the culture without exceeding the maximum concentration specified. In one aspect the APCs are dendritic cells.

It is hypothesized that T cell products that have been expanded in vitro using reduced concentrations of IL-2 as defined above will advantageously require lower doses of IL-2 in vivo in order to persist and engraft.

In one aspect of the invention as described herein said T cell therapy may comprise T cells that have been expanded in the presence of IL-2 at a concentration of less than about 1,000 IU/ml, preferably in the presence of IL-2 at a concentration of about 100 IU/ml.

Interleukin-2 (IL-2)

As described herein, the present invention relates to the administration of low doses of IL-2. Suitable sources of IL-2 according to the invention will be known to those of skill in the art.

The term IL-2 refers to the T cell growth factor known as interleukin-2 and includes all forms of IL-2 including human and mammalian forms, conservative amino acid substitutions, glycoforms, biosimilars and variants thereof. For example, the term IL-2 encompasses human recombinant forms of IL-2 such as Aldesleukin (trade name PROLEUKIN®). Aldesleukin (des-alanyl-I, serine-125 human IL-2) is a nonglycosylated human recombinant form of IL-2 with a molecular weight of approximately 15 kDa. The term IL-2 also encompasses pegylated forms of IL-2, as described in WO 2012/065086.

In one aspect, said IL-2 is administered at a dose of about 1.9 MIU/m2/day, about 1.8 MIU/m2/day, about 1.7 MIU/m2/day, about 1.6 MIU/m2/day, about 1.5 MIU/m2/day, about 1.4 MIU/m2/day, about 1.3 MIU/m2/day, about 1.2 MIU/m2/day, about 1.1 MIU/m2/day, about 1.0 MIU/m2/day, about 0.9 MIU/m2/day, about 0.8 MIU/m2/day, about 0.7 MIU/m2/day, about 0.6 MIU/m2/day, about 0.5 MIU/m2/day, about 0.4 MIU/m2/day, about 0.3 MIU/m2/day or about 0.2 MIU/m2/day.

In one aspect said IL-2 is administered at a dose of about 1.0 MIU/m2/day.

In a further aspect said IL-2 is administered once daily.

In another aspect said IL-2 is administered daily for about 14, 13, 12, 11, 10, 9, 8, 7, 6, 5, 4, 3, 2, or 1 days, preferably 10 days.

In one aspect said IL-2 is administered for less than 14 days, for example about 13, 12, 11, 10, 9, 8, 7, 6, 5, 4, 3, 2, or 1 days, preferably 10 days. In one aspect said IL-2 is administered for not more than 13 days, for example not more than 12, 11, 10, 9, 8, 7, 6, 5, 4, 3, 2, or 1 day.

Said dose of IL-2 may be the same each day.

In one aspect of the invention the total dose of IL-2 administered to said patient does not exceed about 10 MIU/m2.

In one aspect the first dose of said IL-2 is administered on the same day as the T cell therapy.

In one aspect, less than 14 doses of said IL-2 are administered to said patient. For example, 13, 12, 11, 10, 9, 8, 7, 6, 5, 4, 3, 2, or 1 doses of said IL-2 are administered to said patient.

In a preferred aspect, 10 doses of said IL-2 are administered to said patient.

In a further aspect said IL-2 is administered daily on days 0 to 9.

The IL-2 can be administered by any route, including intravenously (IV) and subcutaneously (SC). Low-dose IL-2 is typically given by subcutaneous injection, whereas high-dose IL-2 is generally administered via i.v. infusion. In one particular aspect, the IL-2 is administered subcutaneously.

The invention as described herein may result in reduced toxicity or reduced side effects in the patient due to lower doses of IL-2, on a daily or total basis. That is, the invention may provide a reduction in toxicity or side effects compared with higher doses or longer courses of IL-2.

Lymphodepleting Therapy

Prior to transfer of T cells, patients typically undergo a lymphodepletion therapy. Lymphodepletion treatment improves the efficacy of T cell therapy by reducing the number of endogenous lymphocytes and increasing the serum level of homeostatic cytokines and/or pro-immune factors present in the patient. This creates a more optimal environment for the transplanted T cells to proliferate once administered to the patient. Examples of non-myeloablative lymphodepletion regimens for immunotherapy are disclosed in International Patent Publication No. WO 2004/021995.

In one aspect, the present invention includes administration of a lymphodepleting agent, such as cyclophosphamide and/or fludarabine. In one aspect the invention includes the administration of cyclophosphamide and fludarabine prior to a T cell therapy. The timing of the administration of each component can be adjusted to maximize effect.

As described herein, the day that a T cell therapy is administered may be designated as day 0. The cyclophosphamide and fludarabine may be administered at any time prior to administration of the T cell therapy.

In one aspect, the administration of the cyclophosphamide and fludarabine begins at least seven days, at least six days, at least five days, at least four days, at least three days, at least two days, or at least one day prior to the administration of the T cell therapy.

In another aspect, the administration of the cyclophosphamide and fludarabine may begin at least eight days, at least nine days, at least ten days, at least eleven days, at least twelve days, at least thirteen days, or at least fourteen days prior to the administration of the T cell therapy.

In one aspect, the administration of the cyclophosphamide and fludarabine begins seven days prior to the administration of the T cell therapy. In another aspect, the administration of the cyclophosphamide and fludarabine begins six days prior to the administration of the T cell therapy. In a further aspect, the administration of the cyclophosphamide and fludarabine begins five days prior to the administration of the T cell therapy.

In one particular aspect, administration of the cyclophosphamide begins about seven days prior to the administration of the T cell therapy, and the administration of the fludarabine begins about five days prior to the administration of the T cell therapy. In another aspect, administration of the cyclophosphamide begins about five days prior to the administration of the T cell therapy, and the administration of the fludarabine begins about five days prior to the administration of the T cell therapy.

The timing of the administration of each component can be adjusted to maximize effect. In general, the cyclophosphamide and fludarabine can be administered daily. In some aspects, the cyclophosphamide and fludarabine are administered daily for about two days, for about three days, for about four days, for about five days, for about six days, or for about seven days. In one particular aspect, the cyclophosphamide is administered daily for 2 days, and the fludarabine is administered daily for five days. In another aspect, both the cyclophosphamide and the fludarabine are administered daily for about 3 days.

As described herein, the day the T cell therapy is administered to the patient may be designated as day 0. In some aspects, the cyclophosphamide is administered to the patient on day 7 and day 6 prior to day 0 (i.e., day −7 and day −6). In other aspects, the cyclophosphamide is administered to the patient on day −5, day −4, and day −3. In some aspects, the fludarabine is administered to the patient on day −5, day −4, day −3, day −2, and day −1. In other aspects, the fludarabine is administered to the patient on day −5, day −4, and day −3.

The cyclophosphamide and fludarabine can be administered on the same or different days. If the cyclophosphamide and fludarabine are administered on the same day, the cyclophosphamide can be administered either before or after the fludarabine. In one aspect, the cyclophosphamide is administered to the patient on day −7 and day −6, and the fludarabine is administered to the patient on day −5, day −4, day −3, day −2, and day −1. In another aspect, the cyclophosphamide is administered to the patient on day −5, day −4, and day −3, and the fludarabine is administered to the patient on day −5, day −4, and day −3.

In one particular aspect, the cyclophosphamide and fludarabine are both administered to the patient on day −6, day −5 and day −4.

In certain aspects, cyclophosphamide and fludarabine can be administered concurrently or sequentially. In one aspect, cyclophosphamide is administered to the patient prior to fludarabine. In another aspect, cyclophosphamide is administered to the patient after fludarabine.

The cyclophosphamide and fludarabine can be administered by any route, including intravenously (IV). In some aspects, the cyclophosphamide is administered by IV over about 30 minutes, over about 35 minutes, over about 40 minutes, over about 45 minutes, over about 50 minutes, over about 55 minutes, over about 60 minutes, over about 90 minutes, over about 120 minutes. In some aspects, the fludarabine is administered by IV over about 10 minutes, over about 15 minutes, over about 20 minutes, over about 25 minutes, over about 30 minutes, over about 35 minutes, over about 40 minutes, over about 45 minutes, over about 50 minutes, over about 55 minutes, over about 60 minutes, over about 90 minutes, over about 120 minutes.

As described herein, a T cell therapy may be administered to the patient following administration of cyclophosphamide and fludarabine. In some aspects, the T cell therapy comprises an adoptive cell therapy. In certain aspects, the adoptive cell therapy is selected from tumour-infiltrating lymphocyte (TIL) immunotherapy, autologous T cell therapy, engineered autologous cell therapy (eACT), and allogeneic T cell transplantation. In one particular aspect, the eACT comprises administration of engineered antigen specific chimeric antigen receptor (CAR) positive (+) T cells. In another aspect, the eACT comprises administration of engineered antigen specific T cell receptor (TCR) positive (+) T cells. In some aspects the engineered T cells treat a tumour in the patient.

In one particular aspect, the invention includes a method of conditioning a patient in need of a T cell therapy comprising administering to the patient a dose of cyclophosphamide of about 500 mg/m2/day and a dose of fludarabine of about 60 mg/m2/day, wherein the cyclophosphamide is administered on days −5, −4, and −3, and wherein the fludarabine is administered on days −5, −4, and −3.

In another aspect, the invention includes a method of conditioning a patient in need of a T cell therapy comprising administering to the patient a dose of cyclophosphamide of about 500 mg/m2/day and a dose of fludarabine of about 60 mg/m2/day, wherein the cyclophosphamide is administered on days −7 and −6, and wherein the fludarabine is administered on days −5, −4, −3, −2, and −1. In another aspect, the invention includes a method of conditioning a patient in need of a T cell therapy comprising administering to the patient a dose of cyclophosphamide of about 500 mg/m2/day and a dose of fludarabine of about 30 mg/m2/day, wherein the cyclophosphamide is administered on days −7 and −6, and wherein the fludarabine is administered on days −5, −4, −3, −2, and −1. In another aspect, the invention includes a method of conditioning a patient in need of a T cell therapy comprising administering to the patient a dose of cyclophosphamide of about 300 mg/m2/day and a dose of fludarabine of about 60 mg/m2/day, wherein the cyclophosphamide is administered on days −7 and −6, and wherein the fludarabine is administered on days −5, −4, −3, −2, and −1.

In one aspect the lymphodepleting agent is administered daily for 3 days.

In one aspect the lymphodepleting agent is administered on days −6, −5 and −4 prior to administration of said T cell therapy.

In one aspect cyclophosphamide is administered at a dose of between about 200 mg/m2/day and about 500 mg/m2/day, preferably at a dose of about 200 mg/m2/day, about 250 mg/m2/day, about 300 mg/m2/day, about 350 mg/m2/day, about 400 mg/m2/day, about 450 mg/m2/day or about 500 mg/m2/day. In one aspect said cyclophosphamide is administered at a dose of about 300 mg/m2/day.

In one aspect fludarabine is administered at a dose of between about 20 mg/m2/day and 50 mg/m2/day, preferably at a dose of about 20 mg/m2/day, about 25 mg/m2/day, about 30 mg/m2/day, about 35 mg/m2/day, about 40 mg/m2/day, about 45 mg/m2/day or about 50 mg/m2/day. In one aspect fludarabine is administered at a dose of about 30 mg/m2/day.

In one aspect fludarabine is administered at a dose of about 30 mg/m2 and cyclophosphamide is administered at a dose of about 300 mg/m2 on each of days −6, −5, and −4 prior to cell infusion.

In one aspect the invention provides a method of treating cancer in a patient, comprising administering to the patient:

(i) a lymphodepleting regimen of about 300 mg/m2/day of cyclophosphamide and about 30 mg/m2/day of fludarabine prior to administration of said T cell therapy;

(ii) a single dose of T cell therapy; and

(iii) a dose of IL-2 of about 1.0 MIU/m2/day administered once daily for about 10 days wherein the first dose of said IL-2 is administered on the same day as the T cell therapy.

Cancer

In one aspect the cancer as described herein is selected from lung cancer (small cell, non-small cell and mesothelioma), melanoma, bladder cancer, gastric cancer, oesophageal cancer, breast cancer (e.g. triple negative breast cancer), colorectal cancer, cervical cancer, ovarian cancer, endometrial cancer, kidney cancer (renal cell), brain cancer (eg. gliomas, astrocytomas, glioblastomas), lymphoma, small bowel cancers (duodenal and jejunal), leukaemia, liver cancer (hepatocellular carcinoma), pancreatic cancer, hepatobiliary tumours, germ cell cancers, prostate cancer, merkel cell carcinoma, head and neck cancers (squamous cell), thyroid cancer, high microsatellite instability (MSI-H), and sarcomas.

In one aspect the cancer is selected from melanoma and non-small cell lung cancer (NSCLC).

In one aspect the cancer, such as melanoma or NSCLC, may be metastatic, and/or inoperable and/or recurrent.

Treatment according to the present invention may also encompass targeting circulating tumour cells and/or metastases derived from the tumour.

Subject

In a preferred aspect of the present invention, the subject is a mammal, preferably a cat, dog, horse, donkey, sheep, pig, goat, cow, mouse, rat, rabbit or guinea pig, but most preferably the subject is a human.

As defined herein “treatment” refers to reducing, alleviating or eliminating one or more symptoms or signs of the disease which is being treated, relative to the symptoms prior to treatment.

“Prevention” (or prophylaxis) refers to delaying or preventing the onset of the symptoms of the disease. Prevention may be absolute (such that no disease occurs) or may be effective only in some individuals or for a limited amount of time.

Combination Therapies

The invention as described herein may also be combined with other suitable therapies.

The methods and uses for treating cancer according to the present invention may be performed in combination with additional cancer therapies. In particular, the T cell compositions according to the present invention may be administered in combination with immunotherapy, immune checkpoint intervention, co-stimulatory antibodies, chemotherapy and/or radiotherapy, targeted therapy or monoclonal antibody therapy.

Immune checkpoint molecules include both inhibitory and activatory molecules, and interventions may apply to either or both types of molecule. Immune checkpoint inhibitors include, but are not limited to, PD-1 inhibitors, PD-L1 inhibitors, Lag-3 inhibitors, Tim-3 inhibitors, TIGIT inhibitors, BTLA inhibitors and CTLA-4 inhibitors, for example. Co-stimulatory antibodies deliver positive signals through immune-regulatory receptors including but not limited to ICOS, CD137, CD27 OX-40 and GITR.

Examples of suitable immune checkpoint interventions which prevent, reduce or minimize the inhibition of immune cell activity include pembrolizumab, nivolumab, atezolizumab, durvalumab, avelumab, tremelimumab and ipilimumab.

A chemotherapeutic entity as used herein refers to an entity which is destructive to a cell, that is the entity reduces the viability of the cell. The chemotherapeutic entity may be a cytotoxic drug. A chemotherapeutic agent contemplated includes, without limitation, alkylating agents, anthracyclines, epothilones, nitrosoureas, ethylenimines/methylmelamine, alkyl sulfonates, alkylating agents, antimetabolites, pyrimidine analogs, epipodophylotoxins, enzymes such as L-asparaginase; biological response modifiers such as IFNα, IL-2, G-CSF and GM-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; and drug-conjugates with a chemotherapeutic agent payload.

‘In combination’ may refer to administration of the additional therapy before, at the same time as or after administration of the T cell composition according to the present invention.

In addition or as an alternative to the combination with checkpoint blockade, the T cell composition of the present invention may also be genetically modified to render them resistant to immune-checkpoints using gene-editing technologies including but not limited to TALEN and Crispr/Cas. Such methods are known in the art, see e.g. US20140120622. Gene editing technologies may be used to prevent the expression of immune checkpoints expressed by T cells including but not limited to PD-1, Lag-3, Tim-3, TIGIT, BTLA CTLA-4 and combinations of these. The T cell as discussed here may be modified by any of these methods.

The T cell according to the present invention may also be genetically modified to express molecules increasing homing into tumours and or to deliver inflammatory mediators into the tumour microenvironment, including but not limited to cytokines, soluble immune-regulatory receptors and/or ligands.

Composition

The T cell therapy and/or IL-2 according to the invention as described herein may be provided in the form of a composition.

The composition may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Compositions, according to the current invention, are administered using any amount and by any route of administration effective for preventing or treating a subject. An effective amount refers to a sufficient amount of the composition to beneficially prevent or ameliorate the symptoms of the disease or condition.

The exact dosage is chosen by the individual physician in view of the patient to be treated. Dosage and administration are adjusted to provide sufficient levels of the active agent(s) or to maintain the desired effect in a subject. Additional factors which may be taken into account include the severity of the disease state, e.g., liver function, cancer progression, and/or intermediate or advanced stage of macular degeneration; age; weight; gender; diet, time; frequency of administration; route of administration; drug combinations; reaction sensitivities; level of immunosuppression; and tolerance/response to therapy. Long acting pharmaceutical compositions are administered, for example, hourly, twice hourly, every three to four hours, daily, twice daily, every three to four days, every week, or once every two weeks depending on half-life and clearance rate of the particular composition.

The active agents of the pharmaceutical compositions of embodiments of the invention are preferably formulated in dosage unit form for ease of administration and uniformity of dosage. The expression “dosage unit form” as used herein refers to a physically discrete unit of active agent appropriate for the patient to be treated. The total daily usage of the compositions of the present invention will be decided by the attending physician within the scope of sound medical judgment. For any active agent, the therapeutically effective dose is estimated initially either in cell culture assays or in animal models, potentially mice, pigs, goats, rabbits, sheep, primates, monkeys, dogs, camels, or high value animals. The cell-based, animal, and in vivo models provided herein are also used to achieve a desirable concentration, total dosing range, and route of administration. Such information is used to determine useful doses and routes for administration in humans.

A therapeutically effective dose refers to that amount of active agent that ameliorates the symptoms or condition or prevents progression of the disease or condition. Therapeutic efficacy and toxicity of active agents are determined by standard pharmaceutical procedures in cell cultures or experimental animals, e.g., ED₅₀ (dose therapeutically effective in 50% of the population) and LD₅₀ (dose lethal to 50% of the population). The dose ratio of toxic to therapeutic effects is the therapeutic index, which is expressed as the ratio, LD₅₀/ED₅₀. Pharmaceutical compositions having large therapeutic indices are preferred. The data obtained from cell culture assays and animal studies are used in formulating a range of dosage for human use.

As formulated with an appropriate pharmaceutically acceptable carrier in a desired dosage, the pharmaceutical composition or methods provided herein is administered to humans and other mammals for example topically for skin tumours (such as by powders, ointments, creams, transdermal patches, devices or drops), orally, rectally, mucosally, sublingually, parenterally, intracisternally, intravaginally, intraperitoneally, intravenously, subcutaneously, percutaneously, bucally, sublingually, (intra)ocularly, interosseously or intranasally, depending on preventive or therapeutic objectives and the severity and nature of the cancer-related disorder or condition.

In one aspect the IL-2 as described herein is administered subcutaneously.

Injections of the pharmaceutical composition include intravenous, subcutaneous, intra-muscular, intraperitoneal, or intra-ocular injection into the inflamed or diseased area directly, for example, for esophageal, breast, brain, head and neck, and prostate inflammation.

Liquid dosage forms are, for example, but not limited to, intravenous, ocular, mucosal, pharmaceutically acceptable emulsions, microemulsions, solutions, suspensions, syrups, and elixirs. In addition to at least one active agent, the liquid dosage forms potentially contain inert diluents commonly used in the art such as, for example, water or other solvents; solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols, fatty acid esters of sorbitan, and mixtures thereof. Besides inert diluents, the ocular, oral, or other systemically-delivered compositions also include adjuvants such as wetting agents, emulsifying agents, and suspending agents.

Dosage forms for topical or transdermal administration of the pharmaceutical composition herein include ointments, pastes, creams, lotions, gels, powders, solutions, sprays, inhalants, or patches. The active agent is admixed under sterile conditions with a pharmaceutically acceptable carrier. Preservatives or buffers may be required. For example, ocular or cutaneous routes of administration are achieved with aqueous drops, a mist, an emulsion, or a cream. Administration is in a therapeutic or prophylactic form. Certain embodiments of the invention herein contain implantation devices, surgical devices, or products which contain disclosed compositions (e.g., gauze bandages or strips), and methods of making or using such devices or products. These devices may be coated with, impregnated with, bonded to or otherwise treated with the composition herein.

Transdermal patches have the added advantage of providing controlled delivery of the active ingredients to the eye and body. Such dosage forms can be made by dissolving or dispensing the compound in the proper medium. Absorption enhancers are used to increase the flux of the compound across the skin. Rate is controlled by either providing a rate controlling membrane or by dispersing the compound in a polymer matrix or gel.

Injectable preparations of the pharmaceutical composition, for example, sterile injectable aqueous or oleaginous suspensions are formulated according to the known art using suitable dispersing agents, wetting agents, and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or a suspending medium. For this purpose, bland fixed oil including synthetic mono-glycerides or di-glycerides is used. In addition, fatty acids such as oleic acid are used in the preparation of injectables. The injectable formulations are sterilized prior to use, for example, by filtration through a bacterial-retaining filter, by irradiation, or by incorporating sterilizing agents in the form of sterile solid compositions, which are dissolved or dispersed in sterile water or other sterile injectable medium. Slowing absorption of the agent from subcutaneous or intratumoral injection was observed to prolong the effect of an active agent. Delayed absorption of a parenterally administered active agent is accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release is controlled. Examples of other biodegradable polymers include (poly) orthoesters and (poly)anhydrides. Depot injectable formulations are also prepared by entrapping the agent in liposomes or microemulsions that are compatible with body tissues.

Solid dosage forms for oral administration include capsules, tablets, pills, powders, and granules. In solid dosage forms, the active agent is mixed with at least one inert, pharmaceutically acceptable excipient or carrier such as sodium citrate, dicalcium phosphate, fillers, and/or extenders such as starches, sucrose, glucose, mannitol, and silicic acid; binders such as carboxymethylcellulose, alginates, gelatin, polyvinylpyrrolidinone, sucrose, and acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents, for example, cetyl alcohol and glycerol monostearate; absorbents such as kaolin and bentonite clay; and lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using excipients such as milk sugar as well as high molecular weight PEG and the like. The solid dosage forms of tablets, dragees, capsules, pills, and granules are prepared with coatings and shells such as enteric coatings, release controlling coatings, and other coatings known in the art of pharmaceutical formulating. In these solid dosage forms, the active agent(s) are admixed with at least one inert diluent such as sucrose or starch. Such dosage forms also include, as is standard practice, additional substances other than inert diluents, e.g., tableting lubricants and other tableting aids such as magnesium stearate and microcrystalline cellulose. In the case of capsules, tablets and pills, the dosage forms may also include buffering agents. The composition optionally contains opacifying agents that release the active agent(s) only, preferably in a certain part of the intestinal tract, and optionally in a delayed manner. Examples of embedding compositions include polymeric substances and waxes.

Kit

In one aspect the invention provides a kit comprising a T cell therapy and IL-2, wherein said IL-2 is for administration at a dose of less than about 2.0 MIU/m2/day.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this disclosure.

This disclosure is not limited by the exemplary methods and materials disclosed herein, and any methods and materials similar or equivalent to those described herein can be used in the practice or testing of aspects of this disclosure. Numeric ranges are inclusive of the numbers defining the range.

The headings provided herein are not limitations of the various aspects or aspects of this disclosure which can be had by reference to the specification as a whole. Accordingly, the terms defined immediately below are more fully defined by reference to the specification as a whole.

The term “protein”, as used herein, includes proteins, polypeptides, oligopeptides and peptides.

Other definitions of terms may appear throughout the specification. Before the exemplary aspects are described in more detail, it is to understand that this disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be described, by way of example only, with reference to the following Examples.

### EXAMPLES

### Example 1

Two open-label, multi-centre, phase I/11a studies are being carried out to characterise the safety and clinical activity of autologous, expanded clonal neoantigen-reactive T cells (cNeT) administered intravenously in adult patients with either advanced inoperable or metastatic non-small cell lung cancer (NSCLC) (NCT04032847) or metastatic or recurrent melanoma (NCT03997474).

Tissue Procurement

Tumour and blood samples procured from the patient are shipped to the manufacturing site for further processing. The tumour and blood samples are sequenced and analysed to identify clonal neoantigens. Using this information, clonal neoantigen peptides are subsequently manufactured. Tumour infiltrating lymphocytes (TIL) are isolated from the tumour tissue. The blood sample is used to manufacture dendritic cells which can process and present the clonal neoantigen peptides to the TIL. The isolated and pre-expanded TIL are combined with the dendritic cells which have been pulsed with the clonal neoantigen peptides, and co-cultured with 100 U/ml IL-2. In this way, clonal neoantigen T cells (cNeT) are specifically isolated and expanded. The cNeT cells are harvested and formulated to form ATL001.

Study Treatment

All patients will receive a non-myeloablative lymphodepletion regimen of fludarabine 30 mg/m² i.v. followed by cyclophosphamide 300 mg/m² i.v. on each of Days −6, −5, and −4 prior to cell infusion.

Eligible patients will receive a single intravenous infusion of ATL001. The cell dose to be administered will be ≥1×10⁷ CD3+ cells. The maximum dose in a 30 ml infusion bag is 1×10⁹ CD3+ cells.

Patients will receive 10 doses of IL-2 1 MIU/m² s.c. daily from days 0-9 of the study, starting approximately 3 hours post-infusion.

Patients will stay in hospital over this treatment period.

Post Treatment Assessments

Following discharge from hospital patients will attend study visits on study days 14, 21 and 28 then at week 6, 12, 18 and 24, and then every 12 weeks until week 104. Safety will be assessed by regular assessments of infusion reactions, adverse events, physical examinations, ECOG status, laboratory tests, vital signs, electrocardiograms, and concomitant medication usage. The severity of AEs will be assessed using National Cancer Institute Common Terminology Criteria for Adverse Events (Version 5.0). Clinical activity will be assessed by CT scans every 6 weeks to week 24 and then every 12 weeks.

Study Design

Following consent and screening, eligible patients will initially enter the study for procurement of tumour tissue and blood to manufacture ATL001. Tumour tissue may be procured either before or after receiving standard systemic therapies. While ATL001 is being manufactured, patients will receive standard therapy.

Study Objectives and Outcome Measures

The primary objective of the study is to describe the safety and tolerability of the study product, assessed by the frequency and severity of adverse events (AEs) and serious adverse events (SAEs) following tissue procurement and administration of lymphodepletion agents, ATL001 and IL-2.

The secondary clinical efficacy endpoints include percentage change from baseline in tumour size, objective response rate (ORR), time to response (TTR), duration of response (DoR), disease control rate (CR+PR+ durable SD), progression free survival (PFS) and overall survival (OS). RECIST v1.1 and imRECIST criteria will be applied.

The exploratory objectives of the study include evaluation of the persistence, phenotype and functionality of cNeT cells and possible relationships with clinical outcomes, the evaluation of potential biomarkers of clinical activity and factors affecting response, and the evaluation of factors that may affect the quality of ATL001.

Blood samples are taken from patients at multiple time points before and after ATL001 administration, at days −6 (pre lymphodepletion), 0 (pre administration), 3, 7, 10, 14, 21 and 28 then at 6 weeks, 12 weeks, 18 weeks and 24 weeks then every 3 months until progression. These blood samples will be utilised for a number of different assays including TCR sequencing to track the TCR that were present in the ATL001 product to see if expansion of specific clones can be observed in the blood of the patient. In addition, samples will be taken to allow for detection and analysis of circulating tumour DNA.

Further blood samples will be taken into heparin at each time point. These will be utilised in a whole blood flow cytometry assay to enumerate key immune cells within the blood. PBMC will then be isolated. The PBMC will be then used in several assays: ELISPOT to determine if reactivity to the neoantigen peptides can be detected ex vivo, and intracellular cytokine staining to determine the phenotype of the responding cells. An extended phenotyping panel using flow cytometry will also be used in order to determine the memory phenotype of the T cells (by looking at CD27, CD28 CD45RA and CCR7 expression), any exhaustion markers that may be present (such as CD57, PD-1, TIM3) and a panel that looks at CD25 and FoxP3 expression to determine the number of T regs present.

Results

We have analysed data from the first six patients in the two ongoing clinical trials, three patients with NSCLC and three with melanoma. Patients had received a median of 2.5 lines of therapy prior to receiving cNeT. All had progressive disease at the time of lymphodepletion prior to cNeT infusion and each patient completed their first scheduled scan six weeks post-cNeT infusion to assess tumor size. Data from these six patients has demonstrated a favourable cNeT tolerability profile and provided encouraging initial evidence of cNeT engraftment.

cNeT Tolerability

Overall, the tolerability profile of cNeT was observed to be similar to that of standard TIL products that have not been enriched for cNeT reactivities, with the lymphodepletion regimen accounting for most of the observed higher-grade adverse events, being neutropenia, and febrile neutropenia/neutropenic sepsis. We observed no grade 3 or 4 toxicities reported as causally related to IL-2. We observed two serious adverse events, or SAEs, that were deemed related or possibly related to ATL001. The first was an instance of immune effector cell-associated neurotoxicity syndrome. The event was also deemed potentially related to IL-2. The patient was treated with dexamethasone and tocilizumab and their acute condition improved. The patient, however, subsequently died due to progression of the underlying cancer. The second SAE was a non-specific encephalopathy (grade 1), which led to hospitalization. The episode of encephalopathy responded to corticosteroids and the patient was discharged from the hospital and continued on the trial. Two additional patients subsequently died due to progression of the underlying cancer.

A formal review of safety was conducted by an Independent Data and Safety Monitoring Committee to review the data from these first six patients. The Data and Safety Monitoring Committee recommended that the two clinical trials should continue as planned with no required modifications.

cNeT Activity

We observed stable disease at six-weeks post-dosing in four out of the six patients and progressive disease in two patients. One patient had a reduction in the size of two of their four tumor lesions by approximately 55% and 90%. Engraftment data for our cNeT are currently available from six patients, with evidence of engraftment being observed in three patients, and the highest engraftment observed in the patient who received the highest cNeT dose. It has been observed in prior studies of CAR-T cell therapies that engraftment and expansion of tumor-reactive T cells post infusion is correlated to clinical response. This correlation has not been evaluable with prior TIL therapies due to the lack of routine characterization of the active component of the infused cells, and the associated inability to track the active component post-dosing. Since we characterize our cell product candidates at the level of individual cNeT reactivities, we are able to determine engraftment, peak expansion, and durability of persistence of clonal neoantigen-reactive T cells. An additional benefit of our detailed product characterization is the ability to demonstrate the polyclonality of both the infused product and the engrafted cells. We have identified between two and 28 unique clonal neoantigen reactivities in individual patient cNeT product candidates in both our clinical trials and have demonstrated the presence of the same polyclonal cNeT reactivities following infusion in both patients in whom engraftment was observed.

### Example 2

Patient T-05 enrolled in the melanoma trial with an initial diagnosis of BRAF wild type cutaneous melanoma in 2006. The patient had previously received a three-cycle combination of ipilimumab in 2017, which was discontinued due to toxicity. The patient remained off treatment and had recurrent cutaneous lesions resected in the years following immunotherapy. A soft tissue lesion was excised from the patient's abdomen in February 2020 and was taken forward into cNeT manufacturing.

Intracellular Cytokine Secretion Assay

Intracellular cytokine staining (ICS) is used to assess cNeT cell function (potency) by measuring the ability of the cell population to produce the effector cytokines IFN-γ and/or TNF-α after stimulation with peptides corresponding to patient specific neoantigens. The ICS assay requires 0.1×10⁶ cNeT for seeding and stimulation for 16-18 hour at 37° C., in the presence of the protein transport inhibitors Brefeldin A and Monensin, which prevent release of cytokines from the cell. cNeT are cultured with the following conditions/stimulants:
1. DMSO, Brefeldin A and Monensin as a negative control to show background cytokine production.
2. Staphylococcus enterotoxin B (SEB) as a positive control
3. Long peptide masterpool and Short peptide masterpool corresponding to patient-specific clonal neoantigens reconstituted in DMSO and diluted in water resulting in a final concentration of 0.17 nmols/mL.

Following stimulation, cells are washed and stained with a fixable viability fluorescent dye to enable identification of live cells during analysis. Cells are subsequently fixed, permeabilised, and incubated with fluorescent antibodies specific for the cell surface identification markers CD3, CD4 and CD8 to identify T cells and T cells subsets, and for the intracellular cytokines IFN-γ and TNF-α to identify T cell function in response to stimulus. Flow cytometry (BD FACSLyric or equivalent) is used to acquire a target of 20,000 live CD3+ cells and data is analysed using the acquisition software FACSuite to identify live CD3+ cells and to calculate total cytokine production. Analysis of cytokine production includes both single (IFN-γ or TNF-α) and dual cytokine-producing cells (IFN-γ and TNF-α). Each condition is run in duplicate and the mean of the duplicates is calculated.

EL/Spot Reactivity Assay

PBMCs were isolated from whole blood samples collected using Ficoll-Paque (Merck Life Sciences). On the first day of the assay frozen PBMCs were thawed at 37° C., mixed with complete TexMACS media (Miltenyi Biotec)+1% Penicillin/Streptomycin (Life Technologies) and centrifugated at 450×g for 7 minutes. Cells were resuspended in complete TexMACS media and rested at 37° C., 5% CO₂ for 4-6 hours. After resting, PBMCs were centrifugated at 450×g for 7 minutes and resuspended in complete CTL Test Medium (CTL Europe Gmbh)+1% GlutaMAX (Life Technologies).

Peptides were reconstituted in 100% DMSO (WAK-Chemie Medical Gmbh), diluted 1:5 in water (Life Technologies), before dilution in complete CTL Test Medium.

After resting, 2×10⁵ cells per well were plated in 96-well, pre-coated plate (Human IFN-γ′ Single Colour ELISpot kit, CTL Europe Gmbh) which had been previously washed with 200 μL DPBS (Life Technologies) twice. 100 μL of negative control (0.66% DMSO), positive control (2 μg/mL Staphylococcal enterotoxin B, Merch Life Sciences) or peptides for testing were added to each well at resulting in a final concentration of 0.000165 nmol/μL for short and long peptide masterpools. Plates were incubated at 37° C., 5% CO₂ for 12-16 hours. Detection antibodies and developing solution were added as per manufacturer's instructions before reading on CTL ELISpot plate reader (Bio-Sys Gmbh Bioreader 6000-FO.

TBNK Assay

50 μL whole blood sample was stained with Multitest™ 6-colour TBNK reagent (BD Biosciences) according to manufacturer's instructions. Prior to sample acquisition, 1 μL of 1 μg/mL 4′,6-diamidino-2-phenylindole (DAPI, BD Biosciences), a DNA binding dye, was added to whole blood samples immediately before sample analysis. Samples were acquired on BD FACSLyric™ and analysed using BD FACSuite™ software.

Results

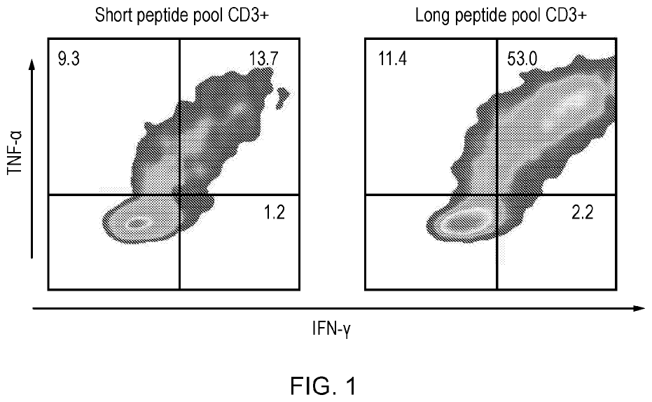

The T cell function of the manufactured product was measured by intracellular cytokine secretion of IFN-γ and TNF-α using flow cytometry (FIG. 1). This shows, along with the multiple reactivities identified by ELISpot analysis, the presence of single as well as multi-functional cytokine-secreting cNeT.

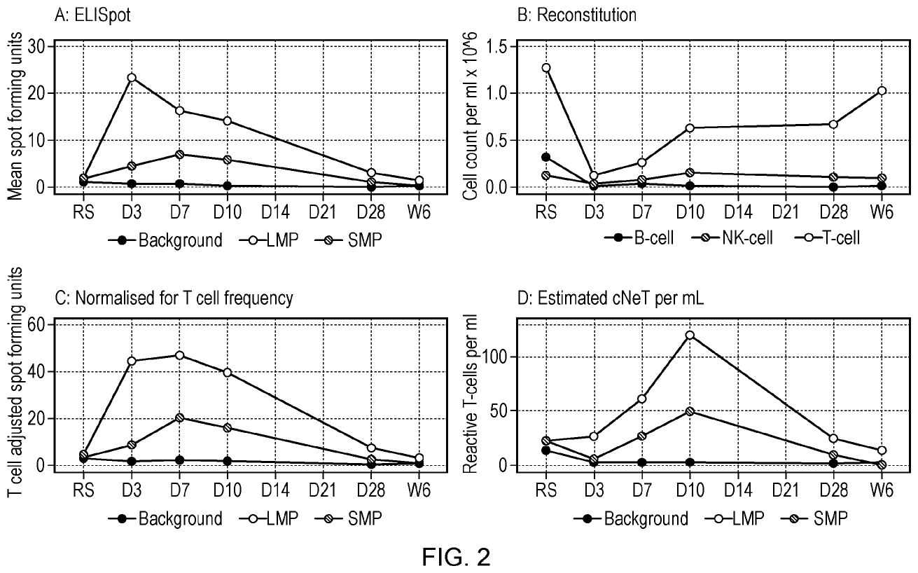

cNeT were tracked pre- and post-dosing in an IFN-γ ELISpot assay using the long and short peptide pools that incorporate the identified clonal mutations (FIG. 2A). Adjusting for the impact of immune system reconstitution observed in TBNK assay (FIG. 2B) allows normalisation for T cell frequency in the ELISpot assay (FIG. 2C) and provides an estimate of the cNeT count/mL in peripheral circulation (FIG. 2D). This shows expansion and detection of cNeT post dosing and provides an estimate of the quantity of reactive T cells in circulation.

1. A method of treating or preventing cancer in a patient, comprising administering to the patient a T cell therapy and a dose of IL-2 of less than about 2.0 MIU/m²/day.

2. The method according to claim 1 wherein said IL-2 is administered at a dose of about 1.9 MIU/m²/day, about 1.8 MIU/m2/day, about 1.7 MIU/m²/day, about 1.6 MIU/m²/day, about 1.5 MIU/m²/day, about 1.4 MIU/m²/day, about 1.3 MIU/m²/day, about 1.2 MIU/m²/day, about 1.1 MIU/m²/day, about 1.0 MIU/m²/day, about 0.9 MIU/m²/day, about 0.8 MIU/m²/day, about 0.7 MIU/m²/day, about 0.6 MIU/m²/day, about 0.5 MIU/m²/day, about 0.4 MIU/m²/day, about 0.3 MIU/m²/day or about 0.2 MIU/m²/day.

3. The method according to claim 1 or claim 2 wherein said IL-2 is administered at a dose of about 1.0 MIU/m²/day.

4. The method according to any one of claims 1 to 3 wherein said IL-2 is administered once daily.

5. The method according to any one of claims 1 to 4 wherein said IL-2 is administered daily for about 14, 13, 12, 11, 10, 9, 8, 7, 6, 5, 4, 3, 2, or 1 days.

6. The method according to claim 5 wherein said IL-2 is administered for less than 14 days.

7. The method according to claim 5 or claim 6 wherein said IL-2 is administered daily for about 10 days.

8. The method according to any one of claims 1 to 7 wherein said dose of IL-2 is the same each day.

9. The method according to any one of claims 1 to 8 wherein the total dose of IL-2 administered to said patient does not exceed about 10 MIU/m².

10. The method according to any one of claims 1 to 9 wherein the first dose of said IL-2 is administered on the same day as the T cell therapy.

11. The method according to any one of claims 1 to 10 wherein said T cell therapy is administered on day 0.

12. The method according to any one of claims 1 to 11 wherein said IL-2 is administered daily on days 0 to 9.

13. The method according to any one of claims 1 to 12 wherein a single dose of T cell therapy is administered to the patient.

14. The method according to any one of claims 1 to 13 wherein a single dose of T cell therapy is administered to the patient on day 0 only.

15. The method according to any one of claims 1 to 14 wherein said IL-2 is administered subcutaneously.

16. The method according to any one of claims 1 to 15 wherein said T cell therapy is selected from adoptive T cell therapy, tumour-infiltrating lymphocyte (TIL) immunotherapy, autologous cell therapy, engineered autologous cell therapy (eACT), and allogeneic T cell transplantation.

17. The method according to any one of claims 1 to 16 wherein said T cell therapy comprises T cells which target neoantigens.

18. The method according to claim 17 wherein said T cell therapy comprises T cells which target clonal neoantigens.

19. The method according to claim 17 or claim 18 wherein said T cell therapy comprises T cells which have been selectively expanded to target clonal neoantigens.

20. The method according to any one of claims 1 to 19 wherein said T cell therapy comprises T cells that have been expanded in the presence of IL-2 at a concentration of less than about 1,000 IU/ml.

21. The method according to any one of claims 1 to 20 wherein said T cell therapy comprises T cells which express a chimeric antigen receptor or a TCR which specifically binds to a clonal neoantigen or an affinity-enhanced TCR which specifically binds to a clonal neoantigen.

22. The method according to any one of claims 1 to 19 further comprising administering a lymphodepleting agent prior to administration of said T cell therapy.

23. The method according to claim 22 wherein said lymphodepleting agent is administered daily for 3 days.

24. The method according to claim 23 wherein said lymphodepleting agent is administered on days −6, −5 and −4 prior to administration of said T cell therapy.

25. The method according to any one of claims 22 to 24 wherein said lymphodepleting agent is cyclophosphamide and/or fludarabine.

26. The method according to claim 25 wherein cyclophosphamide is administered at a dose of between about 200 mg/m²/day and about 500 mg/m²/day.

27. The method according to claim 26 wherein cyclophosphamide is administered at a dose of about 200 mg/m²/day, about 250 mg/m²/day, about 300 mg/m²/day, about 350 mg/m²/day, about 400 mg/m²/day, about 450 mg/m²/day or about 500 mg/m²/day.

28. The method according to claim 27 wherein cyclophosphamide is administered at a dose of about 300 mg/m²/day.

29. The method according to any one of claims 25 to 28 wherein fludarabine is administered at a dose of between about 20 mg/m²/day and 50 mg/m²/day.

30. The method according to claim 28 wherein fludarabine is administered at a dose of about 20 mg/m²/day, about 25 mg/m²/day, about 30 mg/m²/day, about 35 mg/m²/day, about 40 mg/m²/day, about 45 mg/m²/day or about 50 mg/m²/day.

31. The method according to claim 30 wherein fludarabine is administered at a dose of about 30 mg/m²/day.

32. A method of treating cancer in a patient, comprising administering to the patient:
(i) a lymphodepleting regimen of about 300 mg/m2/day of cyclophosphamide and about 30 mg/m2/day of fludarabine prior to administration of said T cell therapy;
(ii) a single dose of T cell therapy; and
(iii) a dose of IL-2 of about 1.0 MIU/m2/day administered once daily for about 10 days wherein the first dose of said IL-2 is administered on the same day as the T cell therapy.

33. The method according to any one of claims 1 to 32, wherein said cancer is lung cancer (small cell, non-small cell and mesothelioma), melanoma, bladder cancer, gastric cancer, oesophageal cancer, breast cancer (e.g. triple negative breast cancer), colorectal cancer, cervical cancer, ovarian cancer, endometrial cancer, kidney cancer (renal cell), brain cancer (eg. gliomas, astrocytomas, glioblastomas), lymphoma, small bowel cancers (duodenal and jejunal), leukaemia, liver cancer (hepatocellular carcinoma), pancreatic cancer, hepatobiliary tumours, germ cell cancers, prostate cancer, merkel cell carcinoma, head and neck cancers (squamous cell), thyroid cancer, high microsatellite instability (MSI-H), and sarcomas.

34. The method according to any one of claims 1 to 33 wherein said method results in reduced toxicity or reduced side effects in the patient.

35. The method according to any one of claims 1 to 34 wherein said patient is a human.

36. A T cell therapy according to any of the preceding claims for use in the treatment or prevention of cancer in a patient, wherein said T cell therapy is for administration with IL-2, and wherein said IL-2 is for administration at a dose of less than about 2.0 MIU/m²/day.

37. A T cell therapy and IL-2 according to any of the preceding claims for use in the treatment or prevention of cancer in a patient, wherein said IL-2 is for administration at a dose of less than about 2.0 MIU/m²/day.

38. A T cell therapy according to any of the preceding claims for use in the manufacture of a medicament for use in the treatment or prevention of cancer, wherein said T cell therapy is for administration with IL-2, wherein said IL-2 is for administration at a dose of less than about 2.0 MIU/m²/day.

39. IL-2 according to any of the preceding claims for use in the manufacture of a medicament for use in the treatment or prevention of cancer, wherein said IL-2 is for administration in combination with a T cell therapy, wherein said IL-2 is for administration at a dose of less than about 2.0 MIU/m²/day.
